(19) United States
(12) Patent Application Publication
(10) Pub. No.: US 2026/0337145 A1
(43) Pub. Date: Oct. 29, 2026

(54) METHOD FOR PRODUCING 2-O-TRANS-CAFFEOYLHYDROXYCITRIC ACID

(71) Applicants: KIMIGAFUCHI GAKUEN, Kumamoto (JP); S&B FOODS INC., Tokyo (JP); NATIONAL UNIVERSITY CORPORATION EHIME UNIVERSITY, Ehime (JP)

(72) Inventors: Tsuyoshi IKEDA, Kumamoto (JP); Keisuke NAKASHIMA, Kumamoto (JP); Nanami YOSHINO, Tokyo (JP); Shunsuke NAKAMURA, Tokyo (JP); Hiroyuki ONDA, Tokyo (JP); Masahiro KASSAI, Tokyo (JP); Mariko KOIKE, Tokyo (JP); Takuya SUGAHARA, Ehime (JP); Kosuke NISHI, Ehime (JP); Momoko ISHIDA, Ehime (JP)

(73) Assignees: KIMIGAFUCHI GAKUEN, Kumamoto (JP); S&B FOODS INC., Tokyo (JP); NATIONAL UNIVERSITY CORPORATION EHIME UNIVERSITY, Ehime (JP)

(21) Appl. No.: 19/112,396

(22) PCT Filed: Sep. 29, 2023
(86) PCT No.: PCT/JP2023/035789

§ 371 Date: Mar. 17, 2025

(30) Foreign Application Priority Data
Sep. 30, 2022 (JP) 2022-158668

(51) Int. Cl. C07C 67/56

(52) U.S. Cl. CPC C07C 67/56

(57) ABSTRACT

Provided is a method for producing 2-O-trans-caffeoylhydroxycitric acid, the method including a step in which coriander is immersed in a polar solvent to extract 2-O-trans-caffeoylhydroxycitric acid with the polar solvent.

### TECHNICAL FIELD

The present invention relates to a method for producing 2-O-trans-caffeoylhydroxycitric acid. According to the present invention, 2-O-trans-caffeoylhydroxycitric acid can be efficiently produced.

### BACKGROUND ART

It has been reported that 2-O-trans-caffeoylhydroxycitric acid is contained in the leaves of hibiscus (Non-Patent Literature 1).

### CITATION LIST

### Non-Patent Literature

[NON-PATENT LITERATURE 1] NATURAL PRODUCT RESEARCH (Great Britain) 2017, Vol. 31: p 2885-2892

### SUMMARY OF INVENTION

### Technical Problem

However, a method for efficiently producing 2-O-trans-caffeoylhydroxycitric acid has not been known.

Accordingly, an object of the present invention is to provide a method for efficiently producing 2-O-trans-caffeoylhydroxycitric acid.

### Solution to Problem

The inventors of the present invention conducted extensive research on methods for efficiently producing 2-O-trans-caffeoylhydroxycitric acid. As a result, the inventors surprisingly found that 2-O-trans-caffeoylhydroxycitric acid can be efficiently produced by using coriander as a raw material.

The present invention is based on these findings.

Therefore, the present invention relates to the following inventions:
[1] A method for producing 2-O-trans-caffeoylhydroxycitric acid, the method comprising the step of:
immersing coriander in a polar solvent to extract 2-O-trans-caffeoylhydroxycitric acid represented by

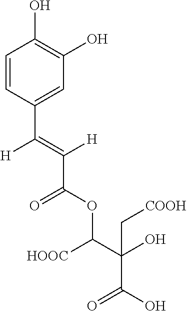 [SMILES: O=C(O)CC(O)(C(=O)O)C(OC(=O)/C=C/c1ccc(O)c(O)c1)C(=O)O]

into the polar solvent,
[2] the method for producing 2-O-trans-caffeoylhydroxycitric acid according to [1], further comprising:
a heat treatment step at a temperature of 65° C. or higher prior to an extraction step,
[3] the method for producing 2-O-trans-caffeoylhydroxycitric acid according to [1] or [2], wherein the extraction step is an extraction step using a polar solvent at a temperature of 65° C. or higher,
[4] the method for producing 2-O-trans-caffeoylhydroxycitric acid according to any one of [1] to [3], wherein the extraction step is an extraction step using an acidic polar solvent,
[5] the method for producing 2-O-trans-caffeoylhydroxycitric acid according to [2], wherein the heat treatment step is blanching,
[6] a coriander extract comprising 2-O-trans-caffeoylhydroxycitric acid, obtained by an extraction method comprising:
immersing coriander in a polar solvent to extract the 2-O-trans-caffeoylhydroxycitric acid represented by

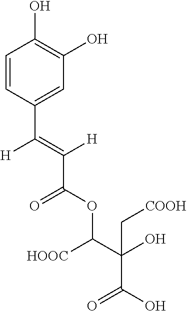 [SMILES: O=C(O)CC(O)(C(=O)O)C(OC(=O)/C=C/c1ccc(O)c(O)c1)C(=O)O]

into the polar solvent,
[7] the coriander extract comprising 2-O-trans-caffeoylhydroxycitric acid according to [6], wherein the method further comprises:
a heat treatment step at a temperature of 65° C. or higher prior to an extraction step,
[8] the coriander extract comprising 2-O-trans-caffeoylhydroxycitric acid according to [6] or [7], wherein the extraction step is an extraction step using a polar solvent at a temperature of 65° C. or higher,
[9] the coriander extract comprising 2-O-trans-caffeoylhydroxycitric acid according to any one of [6] to [8], wherein the extraction step is an extraction step using an acidic polar solvent,
[10] the coriander extract comprising 2-O-trans-caffeoylhydroxycitric acid according to [7], wherein the heat treatment step is blanching,
[11] a method for producing a formulation containing 2-O-trans-caffeoylhydroxycitric acid, the method comprising:
immersing coriander in a polar solvent to extract 2-O-trans-caffeoylhydroxycitric acid represented by

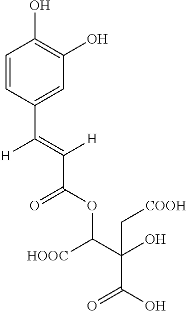 [SMILES: O=C(O)CC(O)(C(=O)O)C(OC(=O)/C=C/c1ccc(O)c(O)c1)C(=O)O]

into the polar solvent, and
formulating the extracted 2-O-trans-caffeoylhydroxycitric acid,
[12] the method for producing the formulation containing 2-O-trans-caffeoylhydroxycitric acid according to [11], further comprising:
a heat treatment step at a temperature of 65° C. or higher prior to an extraction step,
[13] the method for producing the formulation containing 2-O-trans-caffeoylhydroxycitric acid according to [1] or [12], wherein the extraction step is an extraction step using a polar solvent at a temperature of 65° C. or higher,
[14] the method for producing the formulation containing 2-O-trans-caffeoylhydroxycitric acid according to any one of [11] to [13], wherein the extraction step is an extraction step using an acidic polar solvent,
[15] the method for producing the formulation containing 2-O-trans-caffeoylhydroxycitric acid according to [12], wherein the heat treatment step is blanching, and
[16] a composition comprising a coriander in the form of dried state, crushed state, pulverized state, milled state, or powder, wherein the composition is used for extracting 2-O-trans-caffeoylhydroxycitric acid.

### Advantageous Effects of Invention

According to the method for producing 2-O-trans-caffeoylhydroxycitric acid of the present invention, 2-O-trans-caffeoylhydroxycitric acid can be efficiently produced.

In the present specification, the invention is described by dividing into respective aspects, however, the matters described, the definitions of terms, and the embodiments in each aspect can also be applied to other aspects.

### DESCRIPTION OF EMBODIMENTS

### [1] Method for Producing 2-O-Trans-Caffeoylhydroxycitric Acid

The method for producing 2-O-trans-caffeoylhydroxycitric acid according to the present invention comprises immersing coriander in a polar solvent to extract 2-O-trans-caffeoylhydroxycitric acid represented by

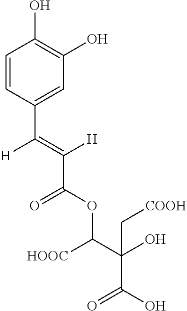 [SMILES: O=C(O)CC(O)(C(=O)O)C(OC(=O)/C=C/c1ccc(O)c(O)c1)C(=O)O]

into the polar solvent.
<<2-O-trans-caffeoylhydroxycitric Acid: Compound A>>

The 2-O-trans-caffeoylhydroxycitric acid (hereinafter sometimes referred to as “Compound A”) is known to be contained in the leaves of hibiscus (Non-Patent Literature 1). However, it has not been reported that Compound A is contained in coriander, which was discovered by the inventors of the present invention. Furthermore, the inventors of the present invention have confirmed that Compound A exhibits anti-allergic activity.

### <<Extraction Step>>

The extraction method in the extraction step of the present invention is a solvent extraction method. It can be carried out in accordance with known solvent extraction methods, except that coriander is used as the raw material and a polar solvent is used as the solvent.

### <<Coriander>>

Coriander is a species in the Apiaceae family and is used as a spice. As a spice, the entire plant body is used, or the fruits and seeds are used as coriander seeds, or the leaves and stems are used as coriander leaves or cilantro.

Compound A is contained in all parts of the coriander plant body. Accordingly, the part of coriander used to produce Compound A in the method of the present invention is not particularly limited and may include the whole plant body, stems, leaves, roots, flowers, fruits, seeds, or any combination of at least two of these. Preferably, the above-ground parts of the plant body, such as stems, leaves, flowers, fruits, and seeds, are used, more preferably leaves are used. A fresh coriander may be used during the extraction operation or a dried (e.g., freeze-dried) coriander may be used. For the extraction operation, it is preferable to process the coriander into a state such as crushed state, pulverized state, milled state, or powder to improve the extraction efficiency.

### <<Polar Solvents>>

The extraction solvent used in the extraction step of the present invention is not particularly limited, as long as Compound A can be extracted into the extract solution. Since Compound A dissolves in polar solvents, the solvent is preferably a polar solvent. Polar solvents include aqueous solvents containing water, polar organic solvents, or mixed solvents containing aqueous or polar organic solvents. Non-polar solvents may be included in the mixed solvents as long as the non-polar solvents do not significantly inhibit the dissolution of Compound A in the polar solvent. The pH of the aqueous solvent is not particularly limited.

Aqueous solvents are not particularly limited as long as the aqueous solvent contains water. For example, water, physiological saline, or buffer solutions can be used. Buffer solutions include phosphate buffer, sodium phosphate buffer, sodium carbonate buffer, citric acid buffer, citric acid-phosphate buffer, acetate buffer, and Tris buffer. The preferable aqueous solvent is water.

Polar organic solvents include alcohols, esters, methylene chloride (dichloromethane), chloroform, diethyl ether, tetrahydrofuran, acetone, acetonitrile, N,N-dimethylformamide, dimethyl sulfoxide, acetic acid, and formic acid. Alcohols include, for example, monohydric alcohols with 1 to 5 carbon atoms, such as methanol, ethanol, propyl alcohol, isopropyl alcohol, and butyl alcohol, and polyhydric alcohols with 2 to 5 carbon atoms, such as 1,3-butylene glycol, propylene glycol, dipropylene glycol, and glycerin.

### (Extraction Temperature)

The temperature of the polar solvent in the extraction step is not particularly limited. Specifically, when the “heat treatment step” described later is performed before the extraction step, the temperature of the extraction (the temperature of the polar solvent) is not limited and can be, for example, −50° C. to 120° C. Preferably, the temperature is −10° C. to 100° C., more preferably 0° C. to 100° C.

It should be noted that “before” the extraction step does not necessarily mean immediately preceding. That is to say, an optional step may be included between the extraction step and the heat treatment step.

When the heat treatment step described later is not performed before the extraction step, the temperature of the extraction (the temperature of the polar solvent) is not particularly limited. However, in this case, the extraction temperature (the temperature of the polar solvent) is preferably 65° C. or higher, more preferably 70° C. or higher, even more preferably 75° C. or higher, and even more preferably 80° C. or higher. The upper limit is not particularly limited, but preferably 100° C. or less.

The extraction operation is preferably performed with stirring or shaking to improve the extraction efficiency. The extraction time can be appropriately determined depending on the part used, such as roots, stems, leaves, flowers, fruits, or seeds. The extraction time can also be appropriately determined based on the state of the coriander. i.e., whether it is fresh or dried, or whether it is processed state, i.e., crushed or powdered. Furthermore, the extraction time can also be appropriately determined based on extraction condition, such as the temperature of the extract solution or the presence or absence of stirring or shaking. The extraction time is typically 1 minute to 72 hours, preferably 10 minutes to 1 hour.

### <<Acidic Polar Solvents>>

In the extraction step of the present invention, acidic aqueous solvents may be used, though not limited to such solvents. In particular, when the “heat treatment step” described later is not performed before the extraction step, using an acidic aqueous solvent can increase the amount of recovery of Compound A. The pH of the acidic polar solvent is not particularly limited, but, for example, it is pH 5 or lower, preferably pH 3 or lower. The acid used to adjust the pH of the solvent to an acidic state can be selected as appropriate depending on the type of polar solvent, however such acids include formic acid, acetic acid, and citric acid. The extraction temperature (polar solvent temperature) of the extraction when using an acidic polar solvent is not particularly limited and may be, for example, −50° C. to 120° C., preferably −10° C. to 100° C., more preferably 0° C. to 100° C. However, in the case of an acidic polar solvent, the extraction temperature may also be 65° C. or higher. The extraction operation and extraction time are as described above.

Acidic polar solvents may further include polar organic solvents. For example, mixed solvents of polar organic solvents and aqueous solvents can increase the recovery rate of Compound A. Polar organic solvents include acetonitrile, alcohols, esters, methylene chloride (dichloromethane), chloroform, diethyl ether, tetrahydrofuran, acetone, N,N-dimethylformamide, or dimethyl sulfoxide.

### <<Heat Treatment Step>>

In the method for producing 2-O-trans-caffeoylhydroxycitric acid of the present invention, the heat treatment step at a temperature of 65′C or higher may be performed before the extraction step, though not limited to such. In particular, when the extraction operation is carried out using a polar solvent of less than 65° C. in the aforementioned extraction process, or when an acidic polar solvent is not used, the amount of Compound A recovered can be increased by carrying out a heat treatment step.

As the heat treatment step, it is not particularly limited as long as heat of 65° C. is applied to the coriander, but preferably the temperature is 70° C. or higher, more preferably 75° C. or higher, and even more preferably 80° C. or higher. The upper limit is not particularly limited, but preferably 100° C. or less.

The heat treatment time is not particularly limited as long as the effects of the present invention can be obtained, but, for example, it may be 10 seconds or more, in certain embodiments, it may be 15 seconds or more, in certain embodiments, it may be 30 seconds or more, in certain embodiments, it may be 1 minute or more, in certain embodiments, it may be 2 minutes or more, in certain embodiments, it may be 3 minutes or more, in certain embodiments, it may be 4 minutes or more, or in certain embodiments, it may be 5 minutes or more. The upper limit of the heat treatment time is not particularly limited as long as 2-O-trans-caffeoylhydroxycitric acid is not degraded by the heat treatment, but for example, it may be 72 hours or less, and in certain embodiments, it may be 1 hour or less, in certain embodiments, it may be 30 minutes or less, in certain embodiments, it may be 10 minutes or less, in certain embodiments, it may be 1 minute or less, in certain embodiments, it may be 30 seconds or less, or in certain embodiments, it may be 15 seconds or less. The heat treatment temperature and heat treatment time can be appropriately combined to achieve the effects of the present invention. For example, if the heat treatment temperature is high, it is possible to obtain the effects of the present invention even if the heat treatment time is short, while if the heat treatment temperature is low, it is possible to obtain the effects of the present invention by increasing the heat treatment time.

Specific heat treatment methods include hot water treatment, steam treatment, hot air treatment, and autoclave treatment. For example, blanching may be one embodiment of hot water treatment or steam treatment. Additionally, autoclave treatment refers to the steam treatment under pressure, where the temperature can be raised to 100° C. or higher. In this specification, “blanching” refers to heating coriander in hot water at a temperature from 70° C. to 100° C. for 10 seconds to 1 minute.

### [2] Cilantro Extract Containing 2-O-Trans-Caffeoylhydroxycitric Acid

The cilantro extract of the present invention is obtained by an extraction method comprising immersing a cilantro in a polar solvent to extract 2-O-trans-caffeoylhydroxycitric acid represented by

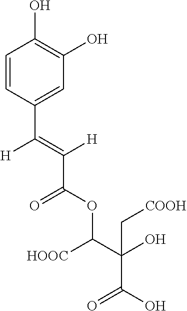 [SMILES: O=C(O)CC(O)(C(=O)O)C(OC(=O)/C=C/c1ccc(O)c(O)c1)C(=O)O]

into the polar solvent.

2-O-trans-caffeoylhydroxycitric acid contained in the extract of the present invention is as described in the item “Method for producing” of the present invention.

The extraction step for obtaining the extract of the present invention is the extraction step described in the section “Method for producing” of the present invention, and the same “polar solvents,” “acidic polar solvents,” and similar solvents can be used. Furthermore, the extraction time and extraction temperature can also be implemented similarly. Additionally, the “heat treatment step” described earlier may be performed before the extraction step to obtain the extract of the present invention.

The cilantro extract may also be contained in a container.

### [3] Method for Producing a Formulation Containing 2-O-Trans-Caffeoylhydroxycitric Acid

A method for producing a formulation containing 2-O-trans-caffeoylhydroxycitric acid, which is one aspect of the present invention, comprises a step of formulating 2-O-trans-caffeoylhydroxycitric acid extracted in the extraction step.

The dosage forms of the formulation include, for example, liquid formulations, pills (pills, tablets), capsules (soft capsules, hard capsules), powders (granules, fine granules), solids, semi-liquids, creams, and pastes.

For formulation methods, known formulation methods can be used, such as powder drying, dissolution, gelation, pasting, concentration, and filtration, either alone or in combination.

### [4] a Composition Comprising a Coriander in the Form of Dried State, Crushed State, Pulverized State, Milled State, or Powder

One aspect of the present invention is a composition comprising coriander in the form of dried state, crushed state, pulverized state, milled state, or powder, which is used for extracting 2-O-trans-caffeoylhydroxycitric acid.

The composition may contain only a coriander in the form of dried state, crushed state, pulverized state, milled state, or powder, or it may contain optional additional components (e.g., desiccants, other spices, etc.). The crushed coriander, pulverized coriander, milled coriander, or powder coriander may also be in a dried state. The composition may be contained in any type of container.

### <<Mode of Action>>

In the production method of the present invention, the ability to efficiently produce (extract or recover) 2-O-trans-caffeoylhydroxycitric acid (Compound A) is attributed to the high content of Compound A in coriander. The inventors of the present invention discovered that coriander contains a large amount of Compound A, enabling the efficient production of 2-O-trans-caffeoylhydroxycitric acid (Compound A) through the present invention.

On the other hand, while the mechanism by which Compound A can be produced in larger quantities using the method of the present invention has not been fully elucidated, it can be hypothesized as follows. However, the present invention is not limited by the following hypothesis.

It is presumed that Compound A contained in coriander may be partially degraded by degradation enzymes present in coriander during the extraction step, thereby reducing the yield. These degradation enzymes, though not particularly limited, are considered to be inactivated by heat or acid. Therefore, it is hypothesized that in the “heat treatment step” of the present invention, or by using a “polar solvent at 65° C. or higher” or an “acidic polar solvent” in the extraction step, the degradation enzymes are inactivated, resulting in an increased recovery rate of Compound A.

### EXAMPLES

The present invention now will be further illustrated by, but is by no means limited to, the following Examples.

### Examples 1 and 2

In these Examples, the heat treatment step was performed, and water (Example 1) or hot water (Example 2) was used as the polar solvent to produce Compound A.

Fresh coriander leaves, stems, and roots were blanched using hot water at 80° C. for 20 seconds, drained, and then frozen at −40° C. The frozen coriander leaves, stems, and roots were freeze-dried for 48 hours. The resulting freeze-dried material was pulverized using a hammer mill and sieved through a 48-mesh screen. One gram of the resulting pulverized material was placed in a test tube, and 5 to 20 mL of water or hot water (100° C.) was added. The test tube was left to stand in an ultrasonic cleaner for 60 minutes. The sample was then centrifuged (2000 rpm, 20 minutes), the supernatant was filtered through filter paper, and the extract was collected. The remaining residue after centrifugation was re-treated with water or hot water (100° C.) and left to stand in an ultrasonic cleaner for another 60 minutes. This extraction procedure was repeated four times to obtain the extract.

The amount of Compound A was quantified using HPLC. The results are shown in Table 1. The content of Compound A is expressed as a weight percentage relative to the pulverized dried raw material.

TABLE 1

| | | Content of |
|---|---|---|
| | Solvent | Compound A (wt %) |
| Example 1 | Water | 2.01% |
| Example 2 | Hot water (100 °C) | 2.02% |

### Examples 3 to 5

In these Examples, the heat treatment time was varied when Compound A was produced.

Fresh cilantro leaves were blanched using hot water at 90° C. for 0 seconds (Example 3), 30 seconds (Example 4), or 60 seconds (Example 5). The blanched leaves were then drained and frozen at −40° C. The frozen cilantro leaves were freeze-dried for 72 hours. The resulting freeze-dried material was pulverized using a coffee mill. One gram of the pulverized material was placed in a test tube, and 5 to 20 mL of hot water (100° C.) was added. The test tube was left to stand in an ultrasonic cleaner for 60 minutes. The sample was then centrifuged (2000 rpm, 20 minutes), and the supernatant was filtered through filter paper to collect the extract. The remaining residue after centrifugation was re-treated with hot water (100° C.) and left to stand in an ultrasonic cleaner for another 60 minutes. This extraction procedure was repeated four times to obtain the extract.

The amount of Compound A was quantified using HPLC. The results are shown in Table 2. The content of Compound A is expressed as a weight percentage relative to the pulverized dried raw material.

TABLE 2

| | Heat | | Content of |
|---|---|---|---|
| | treatment | | Compound A |
| | time | Solvent | (wt %) |
| Example 3 | 0 seconds | Hot water (100 °C) | 1.04% |
| Example 4 | 30 seconds | Hot water (100 °C) | 2.84% |
| Example 5 | 60 seconds | Hot water (100 °C) | 2.05% |

### Examples 6 to 8

In these Examples, Compound A was produced without performing the heat treatment step.

Fresh cilantro leaves and stems were ground using a mortar. A total of 3.97 g of the resulting ground material was placed into a test tube (50 mL), and 40 mL of hot water (100° C.) was added. The mixture was shaken using a shaker for 30 minutes. The sample was centrifuged (3500 rpm, 5 minutes), and 5 mL of the supernatant was left to stand under refrigeration for 3 hours (Example 6), at 40° C. for 3 hours (Example 7), or at room temperature for 2 hours (Example 8). The sample was then centrifuged again (11000 rpm, 1 minute) and filtered using a 0.45 μm filter.

The amount of Compound A was quantified using HPLC. The results are shown in Table 3. The content of Compound A is expressed as a weight percentage relative to the raw ground material.

TABLE 3

| | Heat | | Content of |
|---|---|---|---|
| | treatment | | Compound A |
| | step | Solvent | (wt %) |
| Example 6 | None | Hot water (100 °C) | 0.052% |
| Example 7 | None | Hot water (100 °C) | 0.052% |
| Example 8 | None | Hot water (100 °C) | 0.052% |

### Examples 9 and 10

In these Examples, an autoclave was used for a heat treatment step.

Fresh cilantro leaves and stems were frozen at −40° C. The frozen coriander leaves and stems were freeze-dried for 72 hours. The resulting freeze-dried material was pulverized using a mill. The pulverized material was autoclaved (121° C., 2 atmospheres, 20 minutes). A total of 0.2 g of the pulverized material was placed in a test tube, and 5 mL of hot water (100° C.) (Example 9) or water (Example 10) was added and shaken for 20 minutes. The sample was centrifuged (3000 rpm, 5 minutes), and the supernatant was collected. The residue after centrifugation was treated again with hot water (100° C.) or water and shaken for 20 minutes. This extraction procedure was repeated twice to obtain the extract. The extract was centrifuged (11000 rpm, 1 minute) and filtered using a 0.45 μm filter.

The amount of Compound A was quantified using HPLC. The results are shown in Table 4. The content of Compound A is expressed as a weight percentage relative to the pulverized dried raw material.

TABLE 4

| | Heat | | Content of |
|---|---|---|---|
| | treatment | | Compound A |
| | step | Solvent | (wt %) |
| Example 9 | Autoclave | Hot water (100 °C) | 0.25% |
| Example 10 | Autoclave | Water | 0.27% |

### Examples 11 to 14

In these examples, acidic polar solvents were used to produce Compound A. Fresh cilantro leaves and stems were frozen at −40° C. The frozen cilantro leaves and stems were freeze-dried for 72 hours. The resulting freeze-dried material was pulverized using a mill. A total of 0.1 g of the pulverized material was placed in a test tube, and 5 mL of hot water (100° C.) (Example 11), hot water containing 0.5% formic acid (100° C.) (Example 12), water containing 0.5% formic acid (Example 13), or water containing 0.5% formic acid and 50% acetonitrile (ACN) (Example 14) was added. The mixture was left to stand for 10 minutes in a water bath (95° C. for Examples 11 and 12) or at room temperature (Examples 13 and 14). The samples were then shaken for 20 minutes. The samples were centrifuged (3000 rpm, 5 minutes), and the supernatant was collected. The residue after centrifugation was treated again with the same solvent and shaken for 20 minutes. This extraction procedure was repeated twice to obtain the extract. The extract was centrifuged (11000 rpm, 1 minute) and filtered using a 0.45 μm filter.

The amount of Compound A was quantified using HPLC. The results are shown in Table 5. The content of Compound A is expressed as a weight percentage relative to the pulverized dried raw material.

TABLE 5

| | Heat | | Content of |
|---|---|---|---|
| | Treatment | | Compound A |
| | Step | Solvent | (wt %) |
| Example 11 | None | Hot water | 0.99% |
| Example 12 | None | Hot water with 0.5% | 0.76% |
| | | formic acid | |
| Example 13 | None | Water with 0.5% | 0.78% |
| | | formic acid | |
| Example 14 | None | Water with 1% | 1.00% |
| | | formic acid + ACN | |

### Examples 15 and 16

In these Examples, mixed solvents of water and polar organic solvents were used to produce Compound A.

Fresh cilantro leaves and stems were frozen at −40° C. The frozen cilantro leaves and stems were freeze-dried for 72 hours. The resulting freeze-dried material was pulverized using a mill. A total of 0.1 g of the pulverized material was placed in a test tube, and 5 mL of water containing 50% acetonitrile (ACN) (Example 15) or water containing 50% ethanol (EtOH) (Example 16) was added. The mixture was left to stand at room temperature for 10 minutes and then shaken for 20 minutes. The sample was centrifuged (30) rpm, 5 minutes), and the supernatant was collected. The residue after centrifugation was treated again with the same solvent and shaken for 20 minutes. This extraction procedure was repeated twice to obtain the extract. The extract was centrifuged (11000 rpm, 1 minute) and filtered using a 0.45 μm filter.

The amount of Compound A was quantified using HPLC. The results are shown in Table 6. The content of Compound A is expressed as a weight percentage relative to the pulverized dried raw material.

TABLE 6

| | Heat | | Content of |
|---|---|---|---|
| | treatment | | compound A |
| | step | Solvent | (wt %) |
| Example 15 | None | Water + ACN | 0.25% |
| Example 16 | None | Water + EtOH | 0.49% |

### Examples 17 to 19

In these Examples, Compound A was produced using extraction solvents at temperatures of 70° C. to 90° C. without performing a heat treatment step.

Fresh cilantro leaves and stems were frozen at −40° C. The frozen coriander leaves and stems were freeze-dried for 72 hours. The resulting freeze-dried material was pulverized using a mill. A total of 0.1 g of the pulverized material was placed in a test tube, and 5 mL of hot water (Example 17: 70° C., Example 18: 80° C., Example 19: 90° C.) was added. The test tube was left to stand in a water bath at the same temperature as the added hot water for 10 minutes. It was then shaken for 20 minutes. The sample was centrifuged (3000 rpm, 5 minutes), and the supernatant was collected. The extract was centrifuged again (11000 rpm, 1 minute) and filtered using a 0.45 μm filter.

The amount of Compound A was quantified using HPLC. The results are shown in Table 7. The content of Compound A is expressed as a weight percentage relative to the pulverized dried raw material.

TABLE 7

| | Heat | | Content of |
|---|---|---|---|
| | treatment | | compound A |
| | step | Solvent | (wt %) |
| Example 17 | None | Hot water (70 °C) | 0.25% |
| Example 18 | None | Hot water (80 °C) | 1.21% |
| Example 19 | None | Hot water (90 °C) | 1.29% |

### INDUSTRIAL APPLICABILITY

The production method of the present invention can be used for the production of 2-O-trans-caffeoylhydroxycitric acid.

1. A method for producing 2-O-trans-caffeoylhydroxycitric acid, the method comprising:
immersing Coriandrum sativum in a polar solvent to extract 2-O-trans-caffeoylhydroxycitric acid represented by 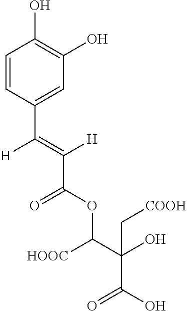 [SMILES: O=C(O)CC(O)(C(=O)O)C(OC(=O)/C=C/c1ccc(O)c(O)c1)C(=O)O]
into the polar solvent.

2. The method for producing 2-O-trans-caffeoylhydroxycitric acid according to claim 1, further comprising:
a heat treatment step at a temperature of 65° C. or higher prior to an extraction step.

3. The method for producing 2-O-trans-caffeoylhydroxycitric acid according to claim 1, wherein the extraction step is an extraction step using a polar solvent at a temperature of 65° C. or higher.

4. The method for producing 2-O-trans-caffeoylhydroxycitric acid according to claim 1, wherein the extraction step is an extraction step using an acidic polar solvent.

5. The method for producing 2-O-trans-caffeoylhydroxycitric acid according to claim 2, wherein the heat treatment step is blanching.

6-10. (canceled)

11. A method for producing a formulation containing 2-O-trans-caffeoylhydroxycitric acid, the method comprising:
obtaining 2-O-trans-caffeoylhydroxycitric acid by the method for producing 2-O-trans-caffeoylhydroxycitric acid according to claim 1, 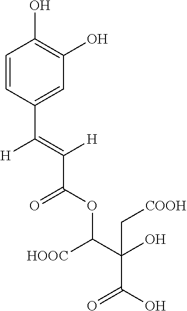 [SMILES: O=C(O)CC(O)(C(=O)O)C(OC(=O)/C=C/c1ccc(O)c(O)c1)C(=O)O]
and
formulating the extracted 2-O-trans-caffeoylhydroxycitric acid into the formulation.

12. The method for producing the formulation containing 2-O-trans-caffeoylhydroxycitric acid according to claim 11, further comprising:
a heat treatment step at a temperature of 65° C. or higher prior to an obtaining step.

13. The method for producing the formulation containing 2-O-trans-caffeoylhydroxycitric acid according to claim 11, wherein the obtaining step is a step using a polar solvent at a temperature of 65° C. or higher.

14. The method for producing the formulation containing 2-O-trans-caffeoylhydroxycitric acid according to claim 11, wherein the obtaining step is a step using an acidic polar solvent.

15. The method for producing the formulation containing 2-O-trans-caffeoylhydroxycitric acid according to claim 12, wherein the heat treatment step is blanching.

16. (canceled)
